(19) United States
(12) Patent Application Publication
(10) Pub. No.: US 2022/0106094 A1
(43) Pub. Date: Apr. 7, 2022

(54) PRINTED PACKAGING MATERIALS

(71) Applicant: The Procter & Gamble Company, Cincinnati, OH (US)

(72) Inventor: Peter Michael Searles, Mason, OH (US)

(21) Appl. No.: 17/495,875

(22) Filed: Oct. 7, 2021

Related U.S. Application Data
(60) Provisional application No. 63/088,618, filed on Oct. 7, 2020.

(51) Int. Cl. B65D 65/42; B41M 5/50

(52) U.S. Cl. CPC B65D 65/42; A61F 13/551; B41M 5/502

(57) ABSTRACT

Printed packaging materials including a novel printed region comprising a light absorbing agent, and packages containing products contained within such packaging materials, and processes for making same are provided.

### FIELD OF THE INVENTION

The present invention relates to novel printed packaging materials, and more particularly to packaging materials comprising a novel printed region comprising a light absorbing agent, and packages comprising products contained within such packaging materials, and processes for making same.

### BACKGROUND OF THE INVENTION

Many products today, especially consumer products, for example personal care products such as sanitary tissue products (paper towels, toilet tissue, facial tissue, and wipes), absorbent products (diapers, training pants, incontinence products, sanitary napkins, and tampons), and hygiene products (bandages and gauze) utilize flexible packaging as their primary packaging and/or as an outerwrap to package multiple products (oftentimes referred to as large count packs), such as cartons, for example facial tissue cartons, and/or toilet paper or paper towel rolls. Such flexible packaging is typically made from films, such as polymeric films, for example thermoplastic polymer films, such as polyolefin-containing films, that contain printed regions, for example one or more printed layers comprising ink. The printed regions, for example printed layers may be on the interior surface of the film and/or on the exterior surface of the film.

In addition to the film packaging comprising printed regions, the products, for example cartons within the film packaging outerwrap may and likely do contain their own printed regions and/or patterns, such as emboss patterns. Thus, the likelihood that the printed regions and/or patterns on the products and/or interior packages, such as cartons contained within the outer film packaging will interfere with the printed regions on the film packaging is high if the opacity of the printed region is not high enough to prevent interference especially when the printed region on the packaging material overlays at least a portion of the printed regions and/or patterns of the products and/or interior packages. Such interference between the different printed regions and/or printed regions and patterns creates an aesthetic nightmare and may be unsightly on the shelves of retail stores and/or online shopping sites as shown in Prior Art FIG. 1. Prior Art FIG. 1 illustrates the problem of interference with a product 10 comprising a packaging material 12 that overwraps one or more products, in this case three interior product packages 14, for example printed cartons such as facial tissue cartons. The packaging material 12 comprising a substrate material 16, for example a transparent or semi-transparent substrate material, such as a film, for example a flexible film. The substrate material 16 comprises a printed region 18 comprising one or more inks. However, the printed region 18 exhibits an opacity insufficient to prevent the interference between the printed patterns on the printed cartons as shown by 20, which are at least partially visible through the printed region 18 of the packaging material 12. In other words, the printed region 18 of the packaging material 12 is not at least 70% opaque as measured according to the Opacity Test Method described herein. Such interference detracts from a consumer's shopping experience and can negatively impact the consumer's desire to by the package that exhibits the interference.

In addition to the film packaging, some packages utilize a label and/or sticker placed on a product's package, for example a bottle. Like the film packaging, the label and/or sticker contain printed regions that also may interfered with by color variation from the contained product or package.

Manufacturers and/or packaging suppliers have attempted to avoid such interference for example between printed regions of inner packages, such as cartons, and printed regions of the outer film packaging and/or labels and/or stickers used with packaging materials, by various failed and/or expensive approaches. They have tried printing opacifying materials, for example materials that reflect light, for example multiple white ink layers (ink containing titanium dioxide) as base layers on the outer film packaging and/or labels and/or stickers making the packaging materials to which their main graphics are printed. Another approach has been to use non-clear films to reduce the transmission of light into and back out of the package thus masking any inner printed regions within the package. These non-clear films, which are oftentimes white, fail to achieve an opacity sufficient to mask the interference for the products and/or sub-packages contained within the non-clear film packaging materials. The non-clear films may contain carbon black within the films to attempt to increase the opacity of the white non-clear films, but this is typically not done due to costs. In addition to the negatives associated with non-clear films discussed above, non-clear films prevent consumers from obtaining additional shopping benefits that a clear, transparent packaging material provides, for example 1) the ability to see the product and/or sub-packages prior to purchase, 2) the ability to see any printed pattern and/or graphics on the product and/or sub-packages prior to purchase, that would be visible during use of the product, and 3) the ability to see how much product remains in the package, for example how much liquid remains in a bottle. Yet another approach has been the use of metallic inks, which are more expensive than non-metallic inks due to the including of metals such as silver flakes within the metallic inks, which also reflect light.

Still yet another approach has been to use darker colors, for example higher pigment levels, on the main graphics printed on the outer film packaging. Problems associated with using darker colors include limited graphic flexibility on the packaging material, for example a darker package requires printed letters and/or logos, such as brand lettering, to be light in color. Light colored lettering on packaging material will look poor unless the packaging material exhibits a high opacity and/or if the brand equity color is lighter, then it will look poor covering product and/or sub-packages unless the packaging material exhibits a high opacity.

All of these above approaches are very expensive as they require the use of high levels of materials, for example by printing multiple base layers and/or using darker inks), expensive materials (metallic inks), and/or the presence of masking materials over substantially the entire surface of the packaging materials (non-clear films). In light of all the failed approaches, only the metallic ink approach since the metal is an opacifier (a light reflecting material) achieves an opacity of greater than 60% at typical packaging printing line speeds, but this approach is also expensive.

In light of the foregoing, the problem with known packaging materials, for example film packaging and/or labels and/or stickers is that they do not achieve opacities of greater than 60% and/or greater than 65% and/or greater than 70% at typically packaging printing line speeds and at lower costs than known technologies to mitigate and/or inhibit the interference between printed regions on inner packages and/or products contained within the packaging materials.

Accordingly, there is a need for printed packaging materials, for example film packaging materials and/or labels and/or stickers that comprise printed regions that exhibit opacities greater than 70% as measured according to the Opacity Test Method described herein.

### SUMMARY OF THE INVENTION

The present invention fulfills the needs described above by providing printed packaging materials, for example film packaging and/or labels and/or stickers that exhibit opacities greater than 70% as measured according to the Opacity Test Method described herein, packages made from such packaging materials, products packaged within such packaging materials, and methods for making such packaging materials.

One solution to the problem identified above is to utilize technologies to primarily absorb light using light absorbing agents in the printed regions of the packaging materials such as film packaging and/or labels and/or stickers rather than using technologies, such as opacifiers, like metallic inks, to primarily reflect light as is done currently by using light reflecting agents like opacifying agents in the printed regions. It has unexpectedly been found that by including one or more light absorbing agents in a printed region of packaging materials, for example on the packaging material's substrate material, such as film packaging and/or labels and/or stickers, the printed region of the printed packaging material exhibits an opacity of at least 70% (in one example at least 70% to about 100%, in another example greater than 70%) as measured according to the Opacity Test Method described herein.

In one example of the present invention, a packaging material (printed packaging material), for example a film such as film packaging material, and/or a label and/or sticker comprising a substrate material comprising a surface, wherein the surface comprises a printed region comprising a light absorbing agent such that the printed region exhibits an opacity of at least 70% (in one example at least 70% to about 100%, in another example greater than 70%) as measured according to the Opacity Test Method described herein is provided.

In another example of the present invention, a package for packaging one or more products, such as cartons and/or rolls and/or powder and/or liquid and/or cream, wherein the package comprises the printed packaging material according to the present invention.

In another example of the present invention, a product packaged within a package according to the present invention.

In even another example of the present invention, a method for making such printed packaging material, wherein the method comprises the steps of:
a. providing a packaging material, for example a substrate material, such as a film and/or label and/or sticker;
b. adding a printed region to a surface of the substrate material;
c. adding a light absorbing agent to the printed region such that a printed packaging material comprising the printed region is formed and wherein the printed region exhibits an opacity of at least 70% (in one example at least 70% to about 100%, in another example greater than 70%) as measured according to the Opacity Test Method described herein is formed is provided.

The present invention provides novel printed packaging materials that overcome the negatives present in current packaging materials, especially those packaging materials that contain secondary packages, such as printed cartons and/or packages, and methods for making same.

### BRIEF DESCRIPTION OF THE DRAWINGS

FIG. 1 is a schematic representation of a prior art product illustrating the problems with current printed packaging materials;

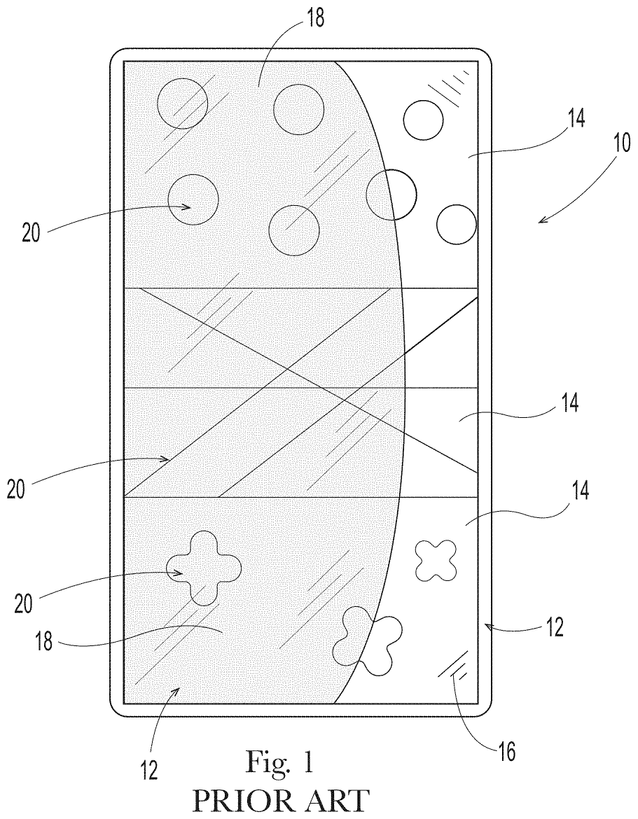

FIG. 2 is a schematic representation of an example of a product comprising the inventive printed packaging material; and

FIG. 3 is a schematic representation of an apparatus for measuring % Compressibility using the % Compressibility Test Method described herein.

### DETAILED DESCRIPTION OF THE INVENTION

Packaging Materials—Film and/or Labels and/or Stickers

The term. “package” or “packaging” herein is intended to mean any container that is meant to be sealed most of the time, especially before the contents are used or taken out, against environmental conditions such as air and/or moisture. The package includes rigid containers and flexible containers, and any conventional method can be used for forming packages from thermoplastics. For rigid containers such as bottles, sealable cartons, storage tanks especially for water, chemicals, fuels and solvents, cosmetic jars, barrels, and drums, the substrate material, for example thermoplastic film of the present disclosure may be used as a shrink sleeve or a layer thereof on the rigid containers, for example shrink sleeve labels, pressure sensitive labels, films 1 aminated with paperboard of cartons. For substrate materials, for example flexible substrate materials used in flexible packaging materials, such as bags or pouches, they may be made directly from the substrate material of the present invention, for example a flexible film.

In some instances, an opaque package with one or more windows (i.e., a transparent translucent portion in the opaque package) is desired, for example when consumers expect to see contents or certain parts of contents in the package or it might visual aesthetics. Such package or at least one layer of such package may be made from the substrate material of the present invention, for example a film. In such a case, one or more portions in the substrate material are windows, through which contents beneath the substrate material and/or within the package are visible.

The packaging materials of the present invention comprise a substrate material onto which a printed region can be created. The substrate material may be any suitable material so long as the substrate is transparent or semi-transparent. For example the substrate material of the printed packaging material comprises one or more printed regions and may optionally comprise one or more non-printed regions that are transparent or semi-transparent.

In one example, the packaging material comprises a transparent or semi-transparent substrate material having a printed region comprising a light absorbing agent, for example carbon black, such that the printed region exhibits an opacity of at least 70% (in one example at least 70% to about 100%, in another example greater than 70%) as measured according to the Opacity Test Method described herein.

In one example, the printed region comprises a varnish, for example a varnish layer present on an ink layer, for example a colored ink layer, of the printed region, wherein the varnish comprises a light absorbing agent, for example carbon black, such that the printed region exhibits an opacity of at least 70% (in one example at least 70% to about 100%, in another example greater than 70%) as measured according to the Opacity Test Method described herein.

The light absorbing agent may be present in a layer, for example a light absorbing agent layer, within the printed region wherein the layer exhibits a thickness of 50 μm or less and/or 40 μm or less and/or 30 μm or less and/or 20 μm or less. In one example, the liquid absorbing agent layer is present within the printed region at a layer thickness of 50 μm to about 5 μm and/or 40 μm to about 5 μm and/or 40 μm to about 10 μm and/or 40 μm to about 20 μm.

The light absorbing agent may be present in the printed region at a level of at least 1% and/or at least 2% and/or at least 3% and/or at least 4% and/or less than 80% and/or less than 70% and/or less than 60% and/or less than 50% and/or less than 40% and/or less than 30% and/or less than 25% and/or less than 20% and/or less than 15% by weight of the printed region. In one example, the light absorbing agent may be present in the printed region at a level of at least 1% to about 80% and/or at least 2% to about 70% and/or at least 3% to about 70% and/or at least 4% to about 70% by weight of the printed region.

At least one printed region of the packaging material comprises one or more inks, for example a cyan ink, a magenta ink, a yellow ink, and/or a black ink. In one example, the one or more inks comprises a metallic ink. One or more of the inks may comprise a non-metallic ink. In one example, the ink may comprise a white ink.

The printed region may comprise one or more inks present in at least one ink layer in the printed region. In one example, the printed region comprises a first ink layer comprising one or more colored inks and a second ink layer comprising a white ink.

In addition to the first ink layer and the second ink layer, the printed region may further comprise a light absorbing agent layer, for example as a varnish.

The printed region may comprise two or more ink layers, at least one of which is a white ink layer, and a varnish layer comprising a light absorbing agent. In one example, the white ink layer is positioned between the substrate material's surface upon which the printed region resides and the varnish layer to minimize the color shift of the intended artwork of the printed region.

The printed region may comprise two or more layers, for example an ink layer and a light absorbing layer, wherein the ink layer is different from the light absorbing layer.

Within a printed region on the packaging material, an ink layer may be positioned between the substrate material's surface and a light absorbing agent layer.

In one example, the substrate material's surface may further comprise one or more non-printed regions. The non-printed regions, when present, may be transparent and/or semi-transparent. At least one of the non-printed regions, when present, may be adjacent to one or more printed regions.

In one example, the substrate material's surface comprises a printed region comprising a light absorbing agent such that the printed region exhibits an opacity of at least 70% and/or at least 75% and/or at least 80% and/or at least 85% and a non-printed region, for example adjacent to the printed region, that exhibits an opacity less than 60% and/or less than 50% and/or less than 40% and/or less than 30% as measured according to the Opacity Test Method described herein. In one example, the printed region exhibits an opacity of at least 70% to 100% and/or from at least 70% to about 95% and/or from at least 70% to about 90% and/or at least 75% to about 90% as measured according to the Opacity Test Method described herein.

In another example, the substrate material comprises a recyclable material and/or compostable material and/or bio-based material that comprises a printed region, for example a printed region comprising a light absorbing agent, that exhibits an opacity of at least 70% and/or at least 75% and/or at least 80% and/or at least 85% to about 100% as measured according to the Opacity Test Method described herein.

In addition to the light absorbing agent, the printed region may further comprise an opacifying agent so long as the opacity of the printed region is at least 70% (in one example at least 70% to about 100%, in another example greater than 70%) as measured according to the Opacity Test Method described herein. The opacifying agent may comprise titanium dioxide.

The packaging material of the present invention may in the form of a roll of packaging material prior to being formed into a package comprising one or more products. Alternatively, the packaging material may be in a pre-made and/or precursor package form, for example a package wicket.

The packages formed from the printed packaging material may comprise one or more sanitary tissue products, for example one or more cartons, for example printed cartons containing facial tissue, one or more rolls of toilet tissue, for example embossed and/or printed toilet tissue, one or more rolls of paper towels, for example embossed and/or printed paper towels, one or more dry and/or wet wipes packages, and/or one or more cartons, for example printed cartons containing napkins and/or paper towels.

When the sanitary tissue products are in rolls, such as rolls of toilet tissue and/or paper towel, the rolls exhibit a Roll Compressibility of at least 1% and/or at least 2% and/or at least 3% as measured according to the % Compressibility Test Method.

Packages comprising absorbent products, for example baby care products, such as diapers and pants and/or feminine hygiene products, such as feminine hygiene pads and/or tampons, and/or adult incontinence products may be packaged using the printed packaging material of the present invention. In one example one or more soft packs of such products may be packaged in the printed packaging material of the present invention. In one example the absorbent products may be discrete articles and/or may be in multi-article sub-packages. The absorbent products may contain print on them in some examples.

In addition to the above products, the packaging material of the present invention may be used to package beauty care products and/or personal care products, such as facial masks.

In addition to packages comprising sanitary tissue products, the printed packaging material of the present invention, for example labels and/or stickers may be applied to other product packaging, for example detergent products, such as bottles of liquid products, for example bottles of liquid detergents and/or shampoo and/or conditioner products, for example bottles of liquid shampoos and/or conditioners, bottles of motor oil, food grade products, such as bottles of food grade liquids, even bottles of powders, such as food grade powders.

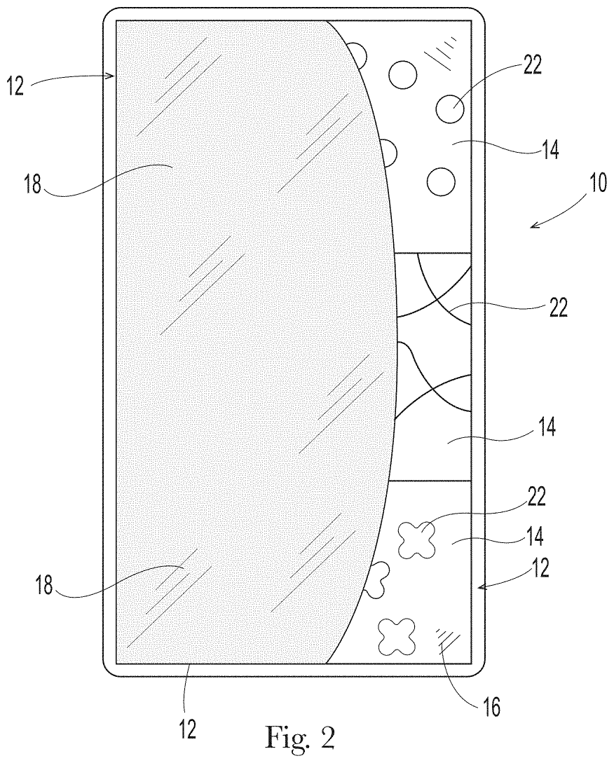

An example of a product comprising a printed packaging material according to the present invention is shown in FIG. 2. FIG. 2 illustrates an example of a product 10 comprising a packaging material 12 that overwraps one or more products, in this case three interior product packages 14, for example printed cartons such as facial tissue cartons. The packaging material 12 comprising a substrate material 16, for example a transparent or semi-transparent substrate material, such as a film, for example a flexible film. The substrate material 16 comprises a printed region 18 comprising one or more inks. The printed region 18 exhibits an opacity of at least 70% (in one example at least 70% to about 100%, in another example greater than 70%) as measured according to the Opacity Test Method described herein that prevents and/or inhibits the interference with the printed patterns 22 present on the interior product packages 14 that are present on the interior product packages 14 below the printed region 18. In one example, the printed region 18 comprises a light absorbing agent, such as carbon black.

### Substrate Material

The substrate material may comprise a film, for example a monolayer or multi-layer film. The film may comprise a polyolefin, for example a polyolefin selected from the group consisting of: polypropylene, polyethylene, copolymers thereof, and mixtures thereof.

The films may be a single layer of film (called a monofilm), a combination of layers that are co-extruded, or a laminate of separately produced layers that are adhered to one another, or an extrusion lamination whereas one layer is extruded onto another previously formed layer(s).

In one example, the films may be foamed films.

In one example the packaging materials of the present invention may comprise a water soluble material, for example polyvinyl alcohol.

Packaging materials for containing and/or holding products, such as consumer products, such as flexible packaging materials, for example film packaging material and/or labels and/or stickers may comprise a substrate material comprising one or more thermoplastic films. Non-limiting examples of suitable thermoplastic films for use in the present invention include polyamide (PA) films, polyethylene (PE) films, polyethylene terephthalate (PET) films, polvmethyl methacrylate (PMMA) films, polypropylene (PP) films, polyurethane (PU) films, polyvinyl acetate (PVA) films, polyvinyl chloride (PVC) films, and mixtures thereof. The thermoplastic films may be found in different grades, for example polyethylene films include the following common grades: high-density polyethylene (HDPE) film, medium-density polyethylene (MDPE) film, linear low-density polyethylene (LLDPE) film, and low-density polyethylene (LDPE) film. The films may be blown or cast, and optionally may be subsequently stretched.

Suitable types of polypropylene (PP) include, but are not limited to: homopolymer isotactic PP and copolymer propylene (coPP). Copolymer propylene (coPP) includes random and block polymers that include ethylene and other alpha-olefin comonomers to form copolymers such as propylene-ethylene block copolymers, propylene-ethylene random copolymers, heterophasic copolypropylene including impact copolypropylene (or “ICP”), as well as any blend thereof. One suitable polypropylene is impact copolymer polypropylene. An example of commercial impact copolymer polypropylene resin is PRO-FAX®7624 available from LyondellBasell (or “LBI”) of Houston, Tex., U.S.A.

Suitable types of polyethylenes (PE) include, but are not limited to: linear low density polyethylene (LLDPE), low density polyethylene (LDPE), medium density polyethylene (MDPE), ethylene vinyl acetate, and ethylene copolymers such as random or multi-block ethylene alpha-olefin. The polyethylenes can be polymerized using any suitable reaction system such as high pressure, slurry, gas phase, and any suitable catalyst system such as Ziegler Natta, constrained geometry, or singlesite metallocene. An example of a suitable commercial polyethylene resin is DOWLEX® 2045G available from the Dow Chemical Company of Midland, Mich., U.S.A. In one example one or more layers of the film may be substantially, or completely free of high density polyethylene (HDPE).

In one example, the substrate material, for example a film, comprises a bio-based content of at least 5% by weight of the film.

In one example, the substrate material, for example a film, comprises recycled material of at least 5% by weight of the film.

The substrate material and/or packaging material of the present invention be and/or comprise renewable materials and/or recyclable materials.

In one example the substrate material and/or packaging material of the present invention are biodegradable and/or compostable.

The substrate material and/or packaging material of the present invention may include a whitening and/or coloring additive added to the polymer resin from which the packaging materials are derived.

The polymer resin used to make the substrate material of the present invention may comprise a traditional petroleum. material-based polyolefin resin, a renewable material-based polyolefin resin, or a blend thereof. Alternatively, the polymer resin may comprise a blend comprising a petroleum material-based polyolefin resin and/or a renewable material-based polyolefin resin mixed with a renewable “bio-new” material that is chemically different to traditional petroleum material-based polyolefin resins. The packaging material may comprise materials, for example polymer resins and/or polymer resin blends, having a total bio-based content of about 10% to about 100% using ASTM D6866-10, method B.

In one example, the substrate material and/or packaging material of the present invention comprises from about 5% to about 99% by weight of a polymer (A). Polymer (A) comprises at least one or possibly more of a low density polyethylene (LDPE), a polar copolymer of polyethylene such as ethylene vinyl acetate (EVA), a linear low density polyethylene (LLDPE), a high density polyethylene homopolymer/high density polyethylene copolymer, a medium density polyethylene, a very low density polyethylene (VLDPE), a plastomer, a polypropylene/copolypropylene/heterophasic polypropylene, polyethylene terephthalate (PET), PLA (e.g., from Natureworks), polyhydroxyalkanoate (PHA), poly(ethylene-2,5-furandicarboxylate) (PEF), cellulose (available from, for example, Innovia), NYLON 11 (i.e., Rilsan® from Arkema), starch (either thermoplastic starch or starch fillers bio-polyesters, (e.g., those made from bio-glycerol, organic acid, and anhydride, as described in U.S. Patent Application No. 2008/0200591, incorporated herein by reference), polybutylene succinate, polyglycolic acid (PGA), and polyvinyl chloride (PVC). At least one of the constituents of polymer (A) is at least partially derived from a renewable resource.

The packaging materials of the present invention, for example films and/or labels and/or stickers, comprise a first surface and a second surface opposite the first surface. In one example, the first surface of the packaging material is an upper surface, the surface that is exposed to the environment and/or consumers, and the second surface is a lower surface, the surface facing the products in the interior package made from the packaging material.

In one example, the substrate materials and/or packaging materials of the present invention are thin, for example thin films, which means that the substrate materials and/or packaging materials exhibit a caliper that is suitable for use in packages such as bags, pouches, labels, stickers, and wraps for consumer goods, such as, for example, calipers from about 10 to about 250 microns.

### Printed Region

The substrate materials and/or packaging materials of the present invention, for example film and/or label and/or sticker comprise a printed region.

The printed region on the substrate material and/or packaging material comprises a light absorbing agent such that the printed region exhibits an opacity of at least 70% and/or at least 75% and/or at least 80% and/or at least 85% and/or 90% and/or 95% and/or at least 70% to about 100% as measured according to the Opacity Test Method described herein.

The printed region may comprise a light absorbing layer comprising a light absorbing agent. The light absorbing layer may exhibit a thickness of 50 μm or less and/or 40 μm or less and/or 30 μm or less and/or 20 μm or less.

The light absorbing agent may comprise carbon black.

In one example, the light absorbing agent may comprise light absorbing organic carbon, also referred to a brown carbon. In still another example, the light absorbing agent may comprise carbon black and brown carbon.

The light absorbing agent may be present in the printed region at a level of at least 1% and/or at least 1.5% and/or at least 2% and/or at least 2.5% and/or at least 3% and/or at least 4% and/or less than 80% and/or less than 70% and/or less than 60% and/or less than 50% and/or less than 40% and/or less than 30% and/or less than 25% and/or less than 20% and/or less than 15% by weight of the printed region (excluding the substrate weight upon which the printed region resides).

In addition to the light absorbing agent, the printed region may further comprise one or more inks. Non-limiting examples of suitable inks for the printed region include metallic inks, non-metallic inks, white ink, and mixtures thereof.

The one or more inks, when present, may be present in one or more ink layers. In one example, the printed region comprises a first ink layer comprising one or more colored inks, for example cyan, magenta, yellow, and black (CMYK). In addition to the first ink layer, the packaging may further comprise one or more additional ink layers, for example a second ink layer comprising a white ink.

An ink layer may be the same layer as the light absorbing layer and/or may be separate from the light absorbing layer.

In one example, the ink layer is sandwich between the packaging material substrate's surface and the light absorbing layer within the printed region.

In addition the packaging material substrate's surface comprising one or more printed regions, the packaging material substrate's surface may further comprise one or more non-printed regions. In one example, at least one of the non-printed regions is transparent and/or semi-transparent.

At least one non-printed region may be adjacent to at least one printed region, for example one printed region comprising a light absorbing agent.

In one example, at least one printed region on the packaging material comprises an opacifying agent, for example titanium dioxide.

### Printing on Substrate Material

Non-limiting examples of methods for printing a printed region on a substrate material of the present invention include flexographic printing and rotogravure printing. Flexographic printing employs a flexible printing plate made of a flexible, elastomeric material, for example a photopolymer. A raised relief image of the indicia to be printed on the package is present on the flexible printer plate. The relief image is coated with ink and then pressed onto the substrate material, such as a film and/or label and/or sticker. Often, one or more flexographic printer plates are positioned on a rotating print cylinder that prints (transfers ink from a bath to an anilox roll, which contains millions of tiny divots to take up the ink from the bath and then the anilox roll contacts the rotating print cylinder containing the flexographic printer plates, which transfers the ink from the anilox roll to the flexographic printer plates , which transfers the ink in the pattern of the flexographic printer plates to the substrate material) on a sheet of substrate material, for example a film, as the substrate material moves beneath the print wheel. Each plate may carry a different type or color ink.

While rotogravure printing relies on impressing the substrate material into cavities of a printer plate where the ink resides, the ink on the flexographic printer plate resides on the raised relief image.

Rotogravure printing uses a printer plate that has an engraved relief image. The printer plate is usually made of metal and is often formed into a cylindrical print roll. Ink is drawn into the engraved image and transferred to the substrate material, for example film. Because both flexographic and rotogravure printing involve contacting the surface of the substrate material with a relief image to transfer the ink to the substrate material, variations in surface texture of the substrate material will impact print quality. Rotogravure presses may have multiple print rolls whereas each print roll can carry a different type or color ink.

Flexographic printing is a method of direct rotary printing that uses a resilient relief image in a plate of rubber or photopolymer to print indicia on a substrate material, for example film and/or label and/or sticker used to make printed packaging materials of the present invention. In many instances, the plate or plates are installed on a rotatable print cylinder that prints on a continuous sheet of substrate material, for example film, as it passes beneath the print cylinder.

In one example, a flat flexographic printer surface is used to print on a substrate material, for example a foamed thin film.

Contact printing processes, such as flexographic and rotogravure printing processes, may be capable of operating effectively on certain substrates at relatively high production rates. However, such contact printing processes have relatively low degrees of flexibility with regard to the ability to change the design of a printed graphic. When utilizing such contact printing methods, changes in graphic designs would often necessitate the shutdown and restart of the entire converting operation. In contrast, some types of printing processes, such as non-contact inkjet printing processes, may provide relatively high degrees of flexibility and ease with regard to the ability to change the design of a printed graphic. In some configurations, a change in graphic design can be implemented by simply inputting commands to a programmed printhead controller to select a desired image to be printed. However, such non-contact printing processes may have limited ability to print graphics at desired print resolutions at relatively high speed production rates.

A printed region on a substrate material and/or packaging material may be obtained by printing compounds such as ink and dyes to form the printed region on a surface of the substrate material and/or packaging material. In one example, the printed region is printed from more than one printing station, for example each station printing a different color of ink.

In one example, the printing system used to print the printed region on the substrate material and/or packaging material comprises two or more printing stations, for example three or more printing stations. Some or all of the printing stations may be arranged on a rotating drum. At least two, for example at least three, of the printing stations may be arranged on the rotating drum. In one example, all the printing stations are arranged on the rotating drum. When one or more printing stations is not arranged on the rotating drum, it may be arranged on an endless rotating horizontal surface.

One or more, for example two or more, or three or more, of the printing stations may be a flexographic printing station. One or more, for example two or more, or three or more, of the printing station may be a digital printing station. The printing system may comprise one or more printing stations selected from digital printing station, flexographic printing station, gravure printing station, rotogravure printing station, lithography, porous and screen printing station, letterpress printing station, tarapography, and combinations thereof. In one example, the printing system comprises at least one flexographic printing station.

One or more printing stations may be used to deliver an over polish varnish (OPY), or other printable materials to the substrate material and/or packaging material. In one example, one or more light absorbing agents may be present in the varnish. One or more printing stations may be used to apply a first layer on the substrate material and/or packaging material to improve the following printing steps, e.g. to improve the adhesion of the compound such as the ink or dye onto the substrate material and/or packaging material. The first layer on the substrate material and/or packaging material and/or the OPV can also be applied h other techniques, such as painting.

### TEST METHODS:

### Opacity Test Method

Opacity of a printed region and/or packaging material and/or package is measured according to ASTM D 589-97, Standard Test Method for Opacity of Paper (15% Diffuse Illuminant A, 89% Reflectance Backing and Paper Backing). The principle is that a material and/or substrate high in opacity will not permit much, if any, light to pass through the material and/or substrate. Whereas a material and/or substrate low in opacity will permit much, if not nearly all, light to pass through the material and/or substrate. Opacity ranges from 0% to 100%

### % Compressibility Test Method

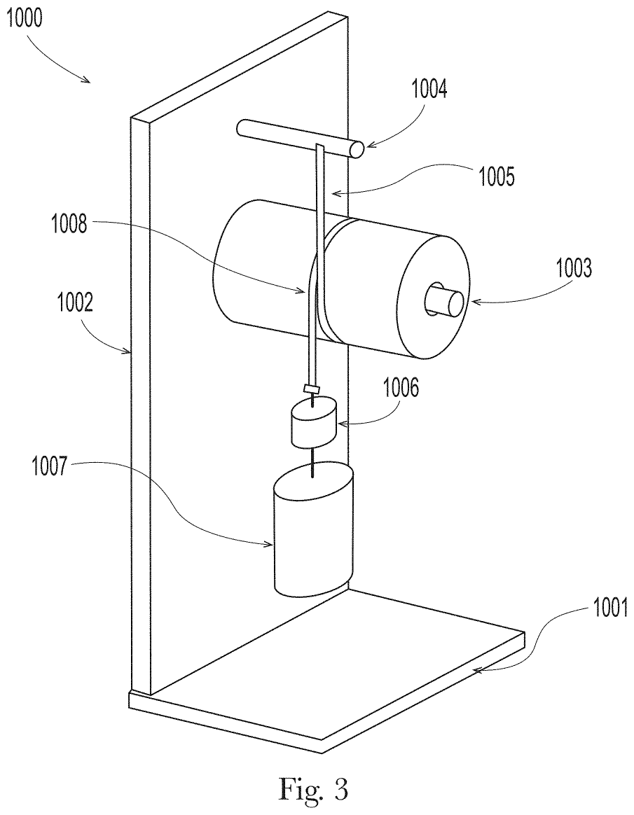

% Roll Compressibility (% Compressibility) is determined using the Roll Diameter Tester 1000 as shown in FIG. 3. It is comprised of a support stand made of two aluminum plates, a base plate 1001 and a vertical plate 1002 mounted perpendicular to the base, a sample shaft 1003 to mount the test roll, and a bar 1004 used to suspend a precision diameter tape 1005 that wraps around the circumference of the test roll. Two different weights 1006 and 1007 are suspended from the diameter tape to apply a confining force during the uncompressed and compressed measurement. All testing is performed in a conditioned room maintained at about 23° C.±2 C.° and about 50%±2% relative humidity.

The diameter of the test roll is measured directly using a Pi® tape or equivalent precision diameter tape (e.g. an Executive Diameter tape available from Apex Tool Group, LLC, Apex, NC, Model No. W606PD) which converts the circumferential distance into a diameter measurement so the roll diameter is directly read from the scale. The diameter tape is graduated to 0.01 inch increments with accuracy certified to 0.001 inch and traceable to NIST. The tape is 0.25 in wide and is made of flexible metal that conforms to the curvature of the test roll but is not elongated under the 1100 g loading used for this test. If necessary the diameter tape is shortened from its original length to a length that allows both of the attached weights to hang freely during the test, yet is still long enough to wrap completely around the test roll being measured. The cut end of the tape is modified to allow for hanging of a weight (e.g. a loop). All weights used are calibrated, Class F hooked weights, traceable to NIST.

The aluminum support stand is approximately 600 mm tall and stable enough to support the test roll horizontally throughout the test. The sample shaft 1003 is a smooth aluminum cylinder that is mounted perpendicularly to the vertical plate 1002 approximately 485 mm from the base. The shaft has a diameter that is at least 90% of the inner diameter of the roll and longer than the width of the roll. A small steel bar 1004 approximately 6.3 mm diameter is mounted perpendicular to the vertical plate 1002 approximately 570 mm from the base and vertically aligned with the sample shaft. The diameter tape is suspended from a point along the length of the bar corresponding to the midpoint of a mounted test roll. The height of the tape is adjusted such that the zero mark is vertically aligned with the horizontal midline of the sample shaft when a test roll is not present.

Condition the samples at about 23° C. ±2 C.° and about 50%±2% relative humidity for 2 hours prior to testing. Rolls with cores that are crushed, bent or damaged should not be tested. Place the test roll on the sample shaft 1003 such that the direction the paper was rolled onto its core is the same direction the diameter tape will be wrapped around the test roll. Align the midpoint of the roll's width with the suspended diameter tape. Loosely loop the diameter tape 1004 around the circumference of the roll, placing the tape edges directly adjacent to each other with the surface of the tape lying flat against the test sample. Carefully, without applying any additional force, hang the 100 g weight 1006 from the free end of the tape, letting the weighted end hang freely without swinging. Wait 3 seconds. At the intersection of the diameter tape 1008, read the diameter aligned with the zero mark of the diameter tape and record as the Original Roll Diameter to the nearest 0.01 inches. With the diameter tape still in place, and without any undue delay, carefully hang the 1000 g weight 1007 from the bottom of the 100 g weight, for a total weight of 1100 g. Wait 3 seconds. Again read the roll diameter from the tape and record as the Compressed Roll Diameter to the nearest 0.01 inch. Calculate percent compressibility to the according to the following equation and record to the nearest 0.1%:

$$\% Compressibility = \frac{( Original Roll Diameter ) - ( Compressed Roll Diameter )}{Original Roll Diameter} \times 100$$

Repeat the testing on 10 replicate rolls and record the separate results to the nearest 0.1%. Average the 10 results and report as the Percent Compressibility to the nearest 0.1%.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm ”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A packaging material comprising a substrate material comprising a surface, wherein the surface comprises a printed region comprising a light absorbing agent such that the printed region exhibits an opacity of at least 70% as measured according to the Opacity.

2. The packaging material according to claim 1 wherein the light absorbing agent is present as a light absorbing agent layer.

3. The packaging material according to claim 1 wherein the light absorbing agent comprises carbon black.

4. The packaging material according to claim 1 wherein the light absorbing agent is present in the printed region at a level of at least 1% by weight of the printed region.

5. The packaging material according to claim 1 wherein the printed region comprises one or more inks.

6. The packaging material according to claim 5 wherein at least one of the one or more inks is present as an ink layer in the printed region.

7. The packaging material according to claim 5 wherein the printed region comprises a first ink layer comprising one or more colored inks and a second ink layer comprising a white ink.

8. The packaging material according to claim 2 wherein the printed region further comprises an ink layer different from the light absorbing layer.

9. The packaging material according to claim 1 wherein the substrate material's surface further comprises a non-printed region.

10. The packaging material according to claim 1 wherein the substrate material comprises a film.

11. The packaging material according to claim 10 wherein the film comprises a bio-based content of at least 5% by weight of the film.

12. The packaging material according to claim 10 wherein the film comprises recycled material of at least 5% by weight of the film.

13. The packaging material according to claim 1 wherein the printed region comprises an opacifying agent.

14. The packaging material according to claim 1 wherein the packaging material is in the form of a roll of packaging material.

15. A package comprising one or more sanitary tissue products contained within the packaging material according to claim 1.

16. The package according to claim 15 wherein the one or more sanitary tissue products are in the form of a roll.

17. The package according to claim 16 wherein at least one of the one or more rolls of sanitary tissue products exhibits a Roll Compressibility of at least 1% as measured according to the % Compressibility Test Method.

18. A package comprising one or more absorbent products contained within package material according to claim 1.

19. The package according to claim 18 wherein the one or more absorbent products are discrete articles.

20. A package comprising a liquid product, wherein the package comprises a packaging material according to claim 1.
